(19) United States
(12) Patent Application Publication
(10) Pub. No.: US 2010/0071724 A1
(43) Pub. Date: Mar. 25, 2010

(54) METHOD OF CLEANING BEVERAGE BOTTLES IN A BEVERAGE BOTTLING PLANT, A METHOD OF CLEANING CONTAINERS IN A CONTAINER FILLING PLANT, AND AN APPARATUS THEREFOR

(76) Inventors: Klaus BAUMGARTNER, Bad Kreuznach (DE); Steffen KAPPEL, Winzenheim (DE); Andreas FAHLDIECK, Idar-Oberstein (DE)

Correspondence Address: NILS H. LJUNGMAN & ASSOCIATES, P. O. BOX 130, GREENSBURG, PA, 15601-0130, US

(21) Appl. No.: 12/408,266

(22) Filed: Mar. 20, 2009

Related U.S. Application Data
(63) Continuation-in-part of application No. PCT/EP2007/007475, filed on Aug. 25, 2007.

(30) Foreign Application Priority Data
Sep. 22, 2006 (DE) 10 2006 044 904.5

(51) Int. Cl. B08B 9/00

(52) U.S. Cl. 134/23; 134/166 R

(57) ABSTRACT

A method of cleaning beverage bottles in a beverage bottling plant, a method of cleaning containers in a container filling plant, and an apparatus therefor. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

This application is a Continuation-In-Part application of International Patent Application No. PCT/EP2007/007475, filed on Aug. 25, 2007, which claims priority from Federal Republic of Germany Patent Application No. 10 2006 044 904.5, filed on Sep. 22, 2006. International Patent Application No. PCT/EP2007/007475 was pending as of the filing date of this application. The United States was an elected state in International Patent Application No. PCT/EP2007/007475.

### BACKGROUND

1. Technical Field

The present application relates to a method of cleaning beverage bottles in a beverage bottling plant, a method of cleaning containers in a container filling plant, and an apparatus therefor.

2. Background Information

Background information is for informational purposes only and does not necessarily admit that subsequently mentioned information and publications are prior art.

The present application relates to a method and an apparatus for the treatment of containers, in one possible embodiment for the upside-down cleaning of bottles, in which at least one sprayer element sprays the interior of the container with a disinfecting or cleaning agent.

The present application relates to a method and an apparatus for the treatment of containers, for example for the upside-down cleaning of bottles, in which at least one sprayer element sprays the interior of the container with a disinfecting or cleaning agent.

The required or desired treatment of containers such as bottles, for example, is conventionally performed on refillable containers made of glass or plastic to remove residual liquids, dirt, dust, plastic particles, etc., from the bottles or containers immediately before or generally before the refilling process. Such cleaning processes are also performed for the initial cleaning of new, unused bottles, for example plastic bottles, before they are filled for the first time. The primary purpose of the treatment is to remove dust, dirt, plastic particles, etc., from the interior of the containers.

In both cases, the containers or bottles are usually placed in an upside-down position and the interior of the bottles or the interior of the containers in this upside-down position is cleaned or “rinsed,” as this process is called in technical jargon, using the disinfecting/cleaning agent. The upside-down position of the bottles or containers essentially ensures or promotes that the (gas or liquid) disinfecting/cleaning agent introduced into the bottles is removed from the bottles and can drain from the containers or bottles via their open mouths under the force of gravity along with any dust, dirt, plastic particles, etc., inside the containers or bottles.

To achieve this effect, the disinfecting/cleaning agent used is generally introduced or sprayed into the interior of the container by means of a sprayer element, the head of which is usually realized in the form of a jet pipe. The disinfecting/cleaning agent is uniformly guided in circulation along the bottom of the container, across the container walls and finally to the mouth, from which the disinfecting and/or cleaning agent exits the individual container or bottle.

In some methods, for example, the disinfection/cleaning is performed with water and/or air and/or steam. The amount of disinfection/cleaning agent consumed is not insignificant, so that in practice the disinfection/cleaning agent is circulated and reused. There are also increasing demands for further conservation of disinfection/cleaning agents to reduce the overall costs of the process. This is where the present application can be of use.

Some solutions have been unable to permanently or substantially permanently resolve these fundamental problems. In fact, some methods for the interior cleaning of reusable returnable bottles involve a sprayer device which is introduced into the containers, which are oriented with their openings or mouths facing downward, and can be moved relative to the container. During the relative movement, the interior wall of the container is sprayed by a high-pressure jet from the sprayer device. For this purpose, the sprayer device is a number of nozzle lances that are fastened to a common lance carrier. The lance carrier can be moved vertically along guides by means of a drive mechanism.

Additionally, some methods for the upside-down treatment of bottles involve a treatment in which a nozzle tube is generally located underneath the upside-down bottle, but the nozzle tube is not inserted into the bottle to the bottom of the container.

### OBJECT OR OBJECTS

One technical problem addressed by the present application is to develop a method of the type described above so that the method can be carried out with a reduced quantity of disinfecting/cleaning agent, thereby reducing the overall costs. An additional object of the present application is an apparatus that is in one possible embodiment suitable for the performance of the method.

### SUMMARY

The present application teaches that this object can be accomplished by a method in which the sprayer element is inserted into the interior of the container, in one possible embodiment into the vicinity of the bottom of the container.

The present application teaches that this object can be accomplished by a method in which the sprayer element is first inserted into the container, which is first sprayed with a disinfecting/cleaning agent, and is then extracted, and is then re-inserted into the interior of the container which is sprayed with a second disinfecting/cleaning agent, whereby different types of bottles can also be processed.

During this process, the sprayer element and the container vary their relative distance from one another. This variability can be achieved by introducing the sprayer element at a variable height into the container, which is conventionally in the upside-down position, and in one possible embodiment until it is in the vicinity of the bottom of the container. The container may maintain its current height during this process. Of course, it is also within the teaching of the present application to fix the spray element in its vertical position and to draw the container down over the sprayer element so that the sprayer element is introduced into the interior of the container, in one possible embodiment into the vicinity of the bottom of the container. Naturally it is also possible to vary the altitude of both the sprayer element and the container, so that ultimately the relative movement between the sprayer element and the container described above is achieved.

The relative distance between the sprayer element and the container is thereby in one possible embodiment variable. This variability can be achieved by introducing the sprayer element at a variable height into the container, which is conventionally in the upside-down position, and in one possible embodiment until it is in the vicinity of the bottom of the container. The container may maintain its current height during this process. Of course, it is also within the teaching of the present application to fix the spray element in its vertical or substantially vertical position and to draw the container down over the sprayer element so that the sprayer element is introduced into the interior of the container, in one possible embodiment into the vicinity of the bottom of the container. Naturally it is also possible to vary the altitude of both the sprayer element and the container, so that ultimately the relative movement between the sprayer element and the container described above is achieved.

In at least one possible embodiment according to the present application, during a cleaning/disinfecting routine, the sprayer element is inserted into the interior of the bottle and extracted from it a plurality of times. This method makes it possible during a single cleaning/disinfecting routine to work with a plurality of different disinfecting/cleaning agents which are introduced separately from one another into the interior of the container. These disinfecting/cleaning agents can be water and/or air, for example. Of course, steam can also be used and the use of steam is also within the teaching of the present application. Of course, steam can also be used and the use of steam is also included.

In at least one possible embodiment, the sprayer element can first be supplied with a first disinfecting/cleaning agent and introduced into the container, or it can be introduced into the container first and then be supplied with the first disinfecting/cleaning agent. The sprayer element then exits the container and is then re-inserted into the interior of the bottle to spray the interior of the bottle with a second disinfecting/cleaning agent.

In this context, the present application basically also makes possible a waterless or liquid-less operation in which the container is cleaned by being purged with a gas, such as air or sterile air, for example, whereby the purging process is performed beginning at the bottom of the bottle and proceeding toward the mouth. In other words, in one possible embodiment, the sprayer element is moved relative to the container during the cleaning/disinfecting routine. In one possible embodiment, the sprayer element is first introduced into the interior of the container into the vicinity of the bottom of the container, and then the removal of the sprayer element begins during the cleaning/disinfecting routine. During this process, the interior of the container is sprayed uniformly, beginning at the bottom of the container and continuing to the mouth.

In this context, the present application basically also makes possible a waterless or liquid-less operation in which the container is cleaned by being purged, whereby the purging process is performed beginning at the bottom of the bottle and proceeding toward the mouth. In other words, in one possible embodiment, the sprayer element is moved relative to the container during the cleaning/disinfecting routine. It has been found to be possible to first introduce the sprayer element into the interior of the container into the vicinity of the bottom of the container, and then to begin the removal of the sprayer element during the cleaning/disinfecting routine. During this process, the interior of the container is sprayed uniformly, beginning at the bottom of the container and continuing to the mouth.

In one possible embodiment, to increase the disinfecting action, the interior of the container can also be irradiated with a UV light source. For this purpose the sprayer element can be equipped with an appropriate UV light source or the discharge aperture of such a UV light source for the sterilization, and can then be introduced into the interior of the container with the sprayer element. Basically, however, it is also possible to introduce the UV light source so that it irradiates the interior of the container from the outside. That process, however, requires or desires the walls of the container to be permeable to UV light.

Under special operating conditions, for example in countries where there is a relative shortage of water and water is correspondingly expensive, in one possible embodiment of the present application spraying with water is eliminated, restricted, and/or minimized entirely or almost entirely. In these cases it is appropriate to replace or at least supplement the cleaning and/or disinfecting action of the water with the sterilizing action of a radiation such as UV radiation, for example. It goes without saying that instead of UV radiation, other suitable types of radiation such as X-rays or infrared radiation can also be used.

It is also within the teaching of the present application to first perform a cleaning with water as a first disinfecting/cleaning agent and then to remove any water film from top to bottom by means of air as the second disinfecting/cleaning agent in the course of a repeated insertion procedure.

In at least one possible embodiment according to the present application, these cleaning routines can be realized if the sprayer element is fastened to a vertically or substantially vertically adjustable actuator. The present application teaches that this vertically or substantially vertically adjustable actuator is realized in two parts. This vertically or substantially vertically adjustable actuator is generally realized in two parts. It comprises essentially a base section and a control section, whereby the base section supports the sprayer element and the control section positions the base section it its relative distance with respect to the container. Overall, the sprayer element can be realized in the form of a jet pipe and can be equipped with an optional wrap-around nozzle on its head. By means of this wrap-around nozzle, the rinsing effect is significantly enhanced. An additional object of the present application is also the apparatus for the treatment of containers, in particular for the upside-down cleaning of bottles, in one possible embodiment for the performance of the method of the present application, with at least one sprayer element which sprays the interior of the container with a disinfecting/cleaning agent, wherein the sprayer element and/or the container can be moved to vary their relative distance from each other. Further embodiments of the apparatus are disclosed according to the present application.

The wrap-around nozzle described can also be a multi-channel nozzle in which there are two or more discharge openings for fluids and/or radiation in the vicinity of the tip of the nozzle. In at least one possible embodiment of the present application, each discharge opening is provided with its own supply line for the medium to be discharged or for the radiation to be emitted, whereby this feed line runs at least partly inside the sprayer element.

The result is a method and an apparatus for the treatment of containers, in one possible embodiment for their cleaning in the upside-down position. The method and apparatus described significantly decrease the quantity of disinfecting/cleaning agent required and/or desired compared with other methods. This reduction is due to, among other things, the fact that the sprayer element is moved relative to the container during the cleaning/disinfecting routine and the areas of the internal wall can be uniformly sprayed or irradiated by the nozzle located on the head, which in one possible embodiment rotates.

The present application therefore employs a direct spraying of the respective interior wall of the container and does not depend on the fact that the disinfection/cleaning agent will dissolve any dirt particles as it travels under the force of gravity along the interior wall of the container from the bottom of the container to the mouth and carry these dirt particles out with it. It thereby becomes possible to significantly reduce the overall quantity of disinfecting/cleaning agent. Consequently, the drainage time is also reduced if the process is conducted with a liquid disinfecting/cleaning agent, and achieves a more efficient rinsing effect as a result of the use of the optional wrap-around nozzle or rotating nozzle.

The above-discussed embodiments of the present invention will be described further herein below. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

The present application is explained in greater detail below on the basis of a single possible embodiment which is illustrated in the accompanying drawing, in which:

FIG. 1 is a schematic side view of the apparatus according to the present application;

FIG. 1A shows one possible embodiment of the present application;

FIG. 1B shows the apparatus of the present application in used with a bottle of a varying size;

FIG. 2 is a plan view of the apparatus for the treatment of containers overall;

FIGS. 3A to 3D illustrate different phases during the cleaning;

FIGS. 4A to 4D show a portion of the apparatus of the present application in use with bottles or containers of varying sizes; and

FIG. 5 shows schematically the main components of one possible embodiment example of a system for filling containers, in one possible embodiment, a beverage bottling plant for filling bottles with at least one liquid beverage, in which system or plant could possibly be utilized with at least one aspect, or several aspects, of the embodiments disclosed herein.

### DESCRIPTION OF EMBODIMENT OR EMBODIMENTS

The figures illustrate an apparatus for the treatment of containers 1, in this case for the upside-down cleaning of bottles 1. Basically, the apparatus can also be used for containers 1 other than bottles 1, and is not necessarily used to clean the bottles 1 or may not be desired to clean the bottles 1. The apparatus illustrated has, in its basic construction, a sprayer element 2 which in the illustrated embodiment is realized non-restrictively in the form of a jet pipe 2. By means of the sprayer element or jet pipe 2, the interior of the individual container or bottle 1 can be sprayed with a disinfecting/cleaning agent 3, as indicated.

In at least one possible embodiment of the present application, the sprayer element or jet pipe 2 and/or the container 1 or bottle 1 are realized so that they can be moved to adjust the relative distance A between them. This distance A is measured, in the context of the illustrated possible embodiment, on the basis of the distance between a head end of the sprayer element or jet pipe 2 and the bottom 1a of the bottle 1.

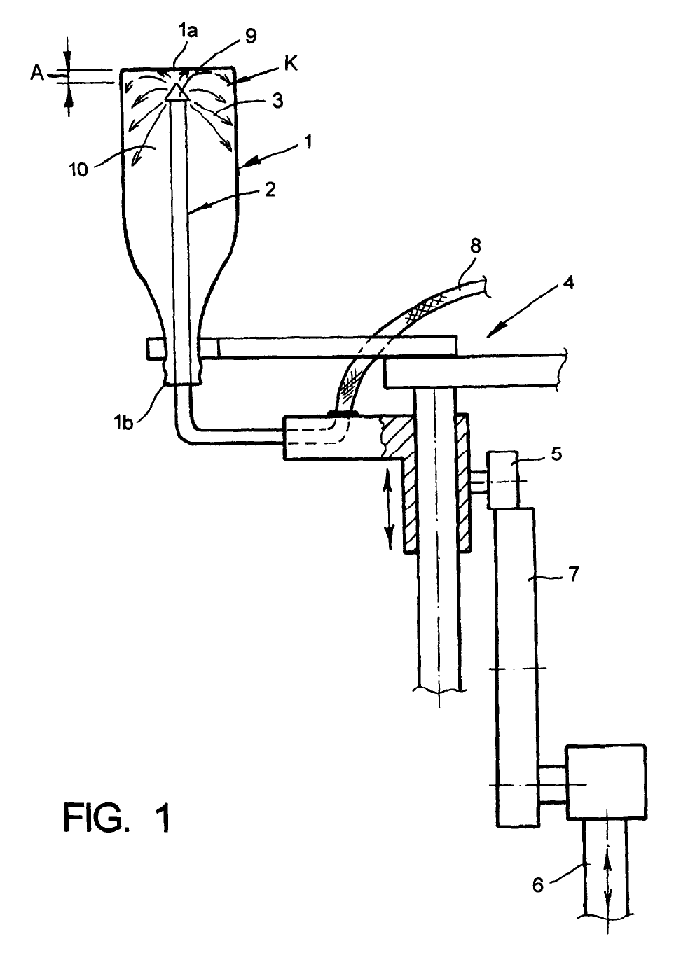

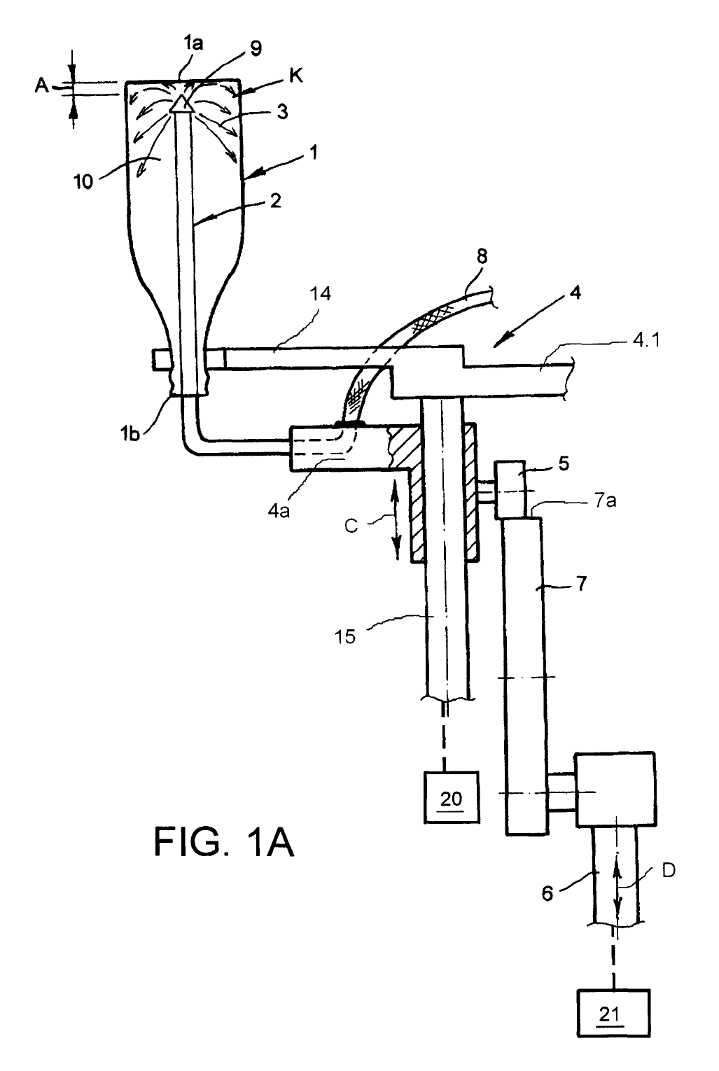

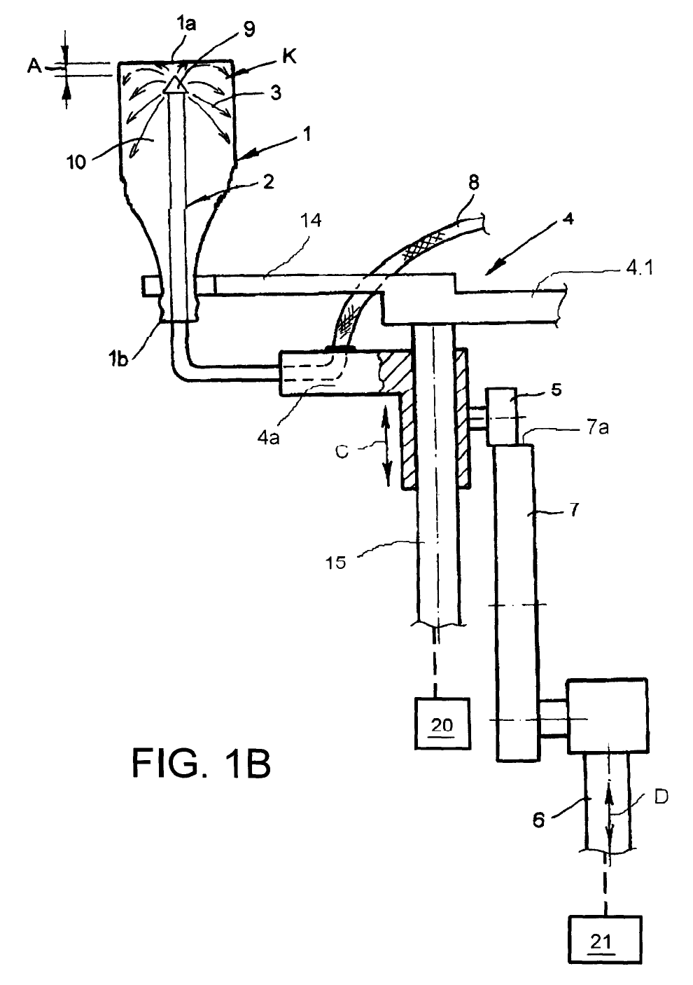

To vary the above-mentioned distance A, the sprayer element 2 in the illustrated possible embodiment has a variable-height actuator device 4, 5, 6, 7. The variable-height actuator device 4, 5, 6, 7 illustrated in FIG. 1 comprises essentially a base section 4, 5 and a control section 6, 7. The base section 4, 5 supports the sprayer element 2, while the control section 6, 7 positions the base section 4, 5 and thus the sprayer element 2 in its relative distance A with respect to the container 1 or the bottle 1.

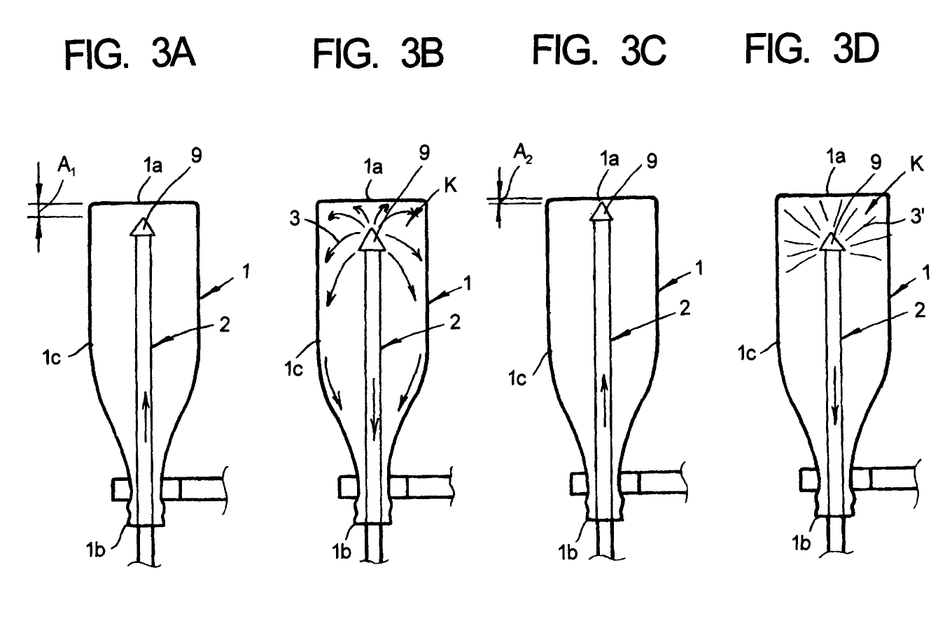

The figures show that the base section 4, 5 has a holding device 4 to which are attached not only the sprayer tube 2 but also a feed line 8 for the delivery of the disinfecting/cleaning agent 3. The base section 4, 5 also has a rotating roller 5 which interacts with a stationary, non-rotating cam system 7 of the control section 6, 7. The cam system 7 is thereby connected to a linear actuator 6 of the control section 6, 7, which makes it possible to adjust the position of the reciprocating movement which is controlled by the cam system 7 and indicated by a double arrow. The reciprocating movement of the roller 5 makes it possible to extend the sprayer element or sprayer tube 2 into the bottle 1 or the interior of the container and remove it from the bottle or container, and to vary its position inside the container, whereby a wide variety of processes can be realized by the appropriate configuration of the cam system 7, as illustrated by way of example and in detail in FIGS. 3A to 3D and explained in greater detail below.

As a result of the mode of operation of the lift adjustment system, in which the height of the reciprocating movement, the magnitude of which is constant or substantially constant, can be set, a wide variety of types of bottles of different lengths can be processed, whereby the sprayer element 2 is inserted into the bottle to the optimum location.

Consequently, the sprayer element or jet pipe 2 is realized so that it can be introduced into the interior of the container, and is conventionally moved during the insertion until it is in one possible embodiment close to the bottom 1a of the container or bottle. In fact, in this position a wrap-around nozzle or multiple-channel nozzle or rotating nozzle 9 which is provided on the top of the sprayer element may correspond to a distance A from the bottom 1a of the bottle which is in the range of a few millimeters, so that in any case the possibility of damage to the bottom 1a of the bottle can be excluded or substantially restricted or minimized. In addition, the disinfecting/cleaning agent 3 that is discharged from the wrap-around nozzle 8 as illustrated is capable of striking the bottom 1a of the bottle. In fact, the disinfecting/cleaning agent 3 exits the wrap-around nozzle 9 in approximately the shape of a spray ball K which is almost closed except for a cone 10.

The distance A between the bottle 1a of the container or bottle 1 and the nozzle 9 of the spray element 2 depends on the viscosity of the cleaning and/or disinfecting agent 3. For example, a highly viscous fluid would produce a spray ball K with a small circumference. On the other hand, a fluid with a low viscosity would produce a spray ball K with a large circumference. A spray element 2 producing a spray ball K with a large circumference could be inserted into the bottle 1 with a longer distance A from the bottom 1a of the bottle 1 and still have the cleaning or disinfecting agent 3 strike the bottle 1a. However, a spray element 2 producing a spray ball K with a small circumference is inserted into the bottle 1 with a smaller distance A to allow the cleaning and/or disinfecting agent 3 to strike the bottle 1a of the bottle or container 1.

In FIG. 1A, and in accordance with at least one possible embodiment of the present application, a bottle 1 is held by a bottle holder 14 in the vicinity of the bottle mouth 1b in a rotary treatment machine. The holder 14 is attached to a portion 4.1 of the variable-height actuator device base 4, which is in turn attached to a shaft 15. The shaft is operatively connected to a motor 20, which is configured to move the shaft 15, portion 4.1, and bottle holder 14 in a vertical or substantially vertical direction, as shown by the double-headed arrow C. This vertical or substantially vertical movement allows the bottle 1 to be moved relative to the spray element 2.

Further seen in FIG. 1A, the portion 4a of the variable-height actuator device 4 is configured to move vertically or substantially vertically along the length of the shaft 15. The portion 4.1 is configured as a stop, which prevents, restricts, or minimizes the portion 4a from moving higher than the portion 4.1 physically permits.

The portion 4a is operatively connected to a roller 5. The variable-height actuator device control section 7 is configured as a ring in the rotary treatment machine. The height of the variable-height actuator device control section 7 varies along the circumference of the ring 7, and the roller 5 moves along the top surface 7a of the variable-height actuator device control section 7. This movement allows the spray element 2 to be moved into and out of the mouths 1b of bottles 1. For example, as the roller 5 moves along the top surface 7a and reaches a low height, the portion 4a is lowered along the shaft 15 and the spray element 2 exits a bottle 1. Likewise, as the roller 5 moves along the top surface 7a and reaches a high height, the portion 4a is raised along the shaft 15 and the spray element 2 enters a bottle 1 through the bottle mouth 1b.

The variable-height actuator device control section 6 is operatively connected to a motor 21, which is configured to move the ring 7 in a vertical or substantially vertical direction, as shown with the double-headed arrow D. Through the vertical or substantially vertical movement of the ring 7, which is actuated by the variable-height actuator device control section 6, bottles 1 of varying sizes can be treated by the rotary treatment machine which comprises the apparatus of the present application. For example, moving the ring 7 lower during operation of the rotary treatment machine allows the machine to treat or handle bottles with lesser heights. Moving the ring 7 higher during operation of the rotary treatment machine allows the machine to treat or handle taller bottles with greater heights.

Also indicated in FIG. 1A is the relative movement between the spray element 2 and the bottle or container 1. For example, in at least one possible embodiment, the bottle 1 could be moved vertically or substantially vertically by the bottle holder 14 as the rotary treatment machine rotates and treats bottles 1, and the portion 4a and spraying element 2 are not moved vertically or substantially vertically as the rotary treatment machine rotates and treats bottles 1. In another possible embodiment, the bottle holder 14 does not move vertically or substantially vertically as the rotary treatment machine rotates and treats bottles 1, so the bottle 1 is not moved vertically or substantially vertically, and the portion 4a and spraying element 2 are moved vertically or substantially vertically as the rotary treatment machine rotates and treats bottles 1. In yet another possible embodiment, the bottle holder 14 and the spraying element 2 and portion 4a are both moved vertically or substantially vertically as the rotary treatment machine rotates and treats bottles 1, so that the nozzle 9 reaches the vicinity of the bottom 1a of the bottle 1 more quickly than in the embodiments with one stationary element and one moving element.

As seen in FIG. 1B, the bottle 1 in this embodiment is a bottle 1 of a smaller size than that which is seen in FIGS. 1 and 1A. Because the length of the bottle 1 is shorter, the vertical or substantially vertical distance that either the bottle holder 14 or the portion 4a and spray element 2 travels is lesser than the distance the holder 14 or the portion 4a and spray element 2 travel when treating a longer bottle 1. With a shallower bottle 1, the ring 7 may be moved to a higher height with the variable-height actuator device control section 6 by means of the motor 21. Once the ring 7 is disposed at a higher level to promote optimal operation of the rotary treatment machine, the height of the bottle holder 14, and thereby the height of the bottle 1, can be adjusted as desired by disposing the shaft 15 higher or lower by means of the motor 20.

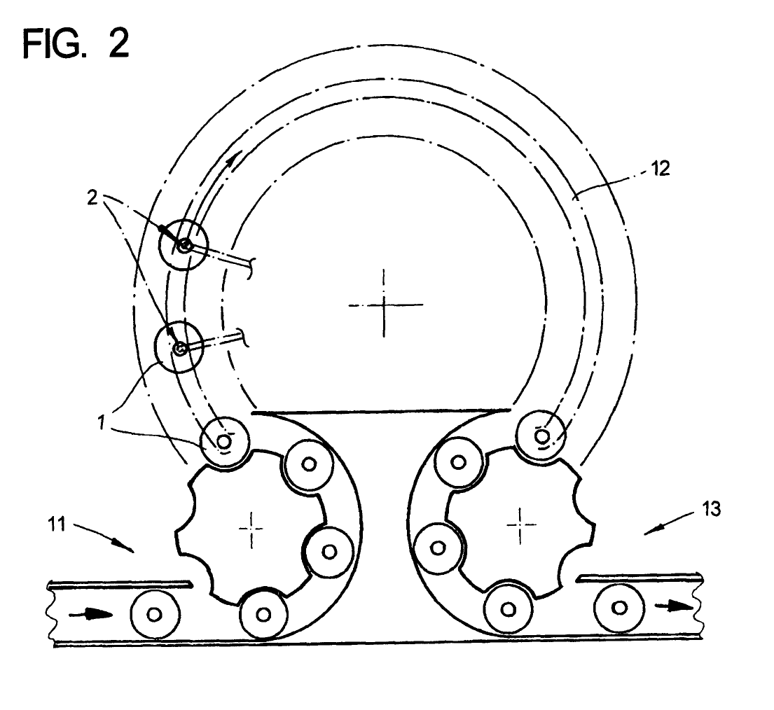

The overview drawing in FIG. 2 shows that numerous devices are located around the periphery of a treatment conveyor 12 as illustrated in FIG. 1. The treatment conveyor 12 works with an inlet conveyor 11 and an outlet conveyor 13. The conveyors 11, 12, 13, may be star-shaped carousel conveyors, although such a realization is not mandatory or may not be desired. In one possible embodiment of the present application, the containers or bottles 1 are transferred into their treatment position in the inlet conveyor 11 and/or the outlet conveyor 13 and/or previously. In other words, the bottles 1 in one possible embodiment reach the treatment conveyors 12 already in their upside-down orientation or position (treatment position). The apparatus described above can thereby be operated at a high speed of rotation of the respective conveyor 11, 12, 13, whereby at the same time the process steps illustrated in detail in FIGS. 3A to 3D can be performed on the path through the treatment conveyor. In other words, the bottles 1 are in one possible embodiment transferred into their overhead positions from the inlet conveyor 11 to the treatment conveyor 12, and the treatment steps illustrated in FIGS. 3A to 3D can be executed as they pass through the treatment conveyor 12. Then the cleaned bottles 1 are transported in their upside-down positions to the exit conveyor 13, which then essentially ensures or promotes that the bottles 1 are (again) in their upright positions with their mouths 1b up.

FIGS. 3A to 3D illustrate various process steps which can be executed, by way of example, with the apparatus described above. First, the figures show that the sprayer element 2 receives a plurality of different disinfecting/cleaning agents 3, 3′ during an entire cleaning/disinfecting routine illustrated in FIGS. 3A and 3D. By way of example, the process step illustrated in FIG. 3B illustrates the spraying of the interior of the container with water 3, while in the process step illustrated in FIG. 3D, a treatment with air 3′ is conducted. In at least one possible embodiment of the present application, the treatment agent 3 is water that is heated to a temperature sufficient to disinfect the container 1. Of course, these specific treatments are not mandatory and/or may not be desired and in this case can be preceded by a treatment with steam or performed in some other sequence.

FIGS. 3A to 3D show that during the cleaning/disinfecting routine illustrated in those figures, the sprayer element 2 enters and exits the interior of the container multiple times. The sprayer element 2 is first supplied with the first disinfecting/cleaning agent, and is introduced into the container. After the removal and repeated introduction into the interior of the container, the bottle is sprayed with the second disinfecting/cleaning agent 3′, which in this case is air. During the individual cleaning processes, the sprayer element 2 is moved relative to the container or the bottle 1 during the cleaning/disinfecting routine. In other words, during individual phases within the cleaning/disinfecting routing, the control cam 7 actuates the stop cam 5 so that the distance A between the top of the jet pipe 2 and the bottom 1a of the bottle is varied.

In the context of the example illustrated in FIGS. 3A to 3D, the jet pipe 2 is first introduced into the bottle 1 into the vicinity of the bottom 1a of the container. During this process, there is not yet any spraying with the disinfecting/cleaning agents 3, 3′. As soon as the desired distance A from the bottom 1a of the bottle is reached, the interior of the container is sprayed with the first disinfecting/cleaning agent 3, in this case water 3. At the same time, the sprayer element 2 is extracted from the interior of the container, so that the first disinfecting/cleaning agent 3 that is discharged from the wrap-around nozzle 9 sprays the interior wall 1c of the container 1 step by step. This process is illustrated in FIG. 3B.

After the removal of the jet pipe 2 from the interior of the bottle 1 or when the mouth 1b is reached, the feed of the first disinfecting/cleaning agent 3 is turned off. The jet pipe illustrated in FIG. 3C is re-inserted into the interior of the bottle 1, and in one possible embodiment until it reaches a predetermined distance A from the bottom 1a of the bottle. The process can thereby be carried out with different distances A, which are identified in the figures by A₁ and A₂. Of course, that depends on the consistency and the pressure of the disinfecting/cleaning agent 3, 3′ fed to the sprayer element 2, and ultimately on the size of the spray ball K which is formed.

In the possible embodiment, as soon as the distance A₂ between the head of the sprayer tube 2 or the wrap-around nozzle 9 that is present on it and the bottom 1a of the bottle is reached, the second disinfecting/cleaning agent 3′ is supplied, e.g. via a separate line that extends into the spray nozzle 2, which in this case is air 3′. By means of the air 3′ which is fed to the sprayer, any water film that may be present in the interior of the container is blown out and a drying process of the interior of the container takes place at the same time. During this process, the wrap-around nozzle 9, and with the nozzle 9 the sprayer tube 2, moves along the interior wall 1c of the container in the direction of the extraction of the sprayer tube 2.

It goes without saying that the individual process steps are specified and can be set by a corresponding actuation of the linear actuator 6 and/or the roller 7 of the control section 6, 7. That may be done according to a variable program, which is stored in a control unit (not shown), which actuates the linear actuator 6 and/or the roller 7 jointly or alternatively—in question.

In the possible embodiment, the linear actuator 6 positions the sprayer tube 2 as a function of the desired size of the bottle 1 to be treated. On the other hand, the independently driven and eccentric control cam 7 essentially ensures or promotes the execution of the individual process steps illustrated in FIGS. 3A to 3D. Of course, it is also possible to realize one drive, namely the linear actuator 6, without additional (eccentric) control cams 7 or with stationary control cams 7. It is also within the framework of the present application to use, instead of the linear actuator 6, an eccentric disc or another type of rotating drive.

The stop cams 5 of the base section 4, 5 must always be actuated or should be actuated so that the sprayer tube 2 takes into consideration different sizes and/or shapes of the containers or bottles 1 as well as the process steps illustrated in FIGS. 3A to 3D. Finally, it is within the framework of the present application to measure the positioning of the sprayer tube 2 by means of a position measurement system or device, the measurement values of which are evaluated by the control unit in the sense of an automatic control system to position or to move the sprayer tube 2 or the wrap-around nozzle 9.

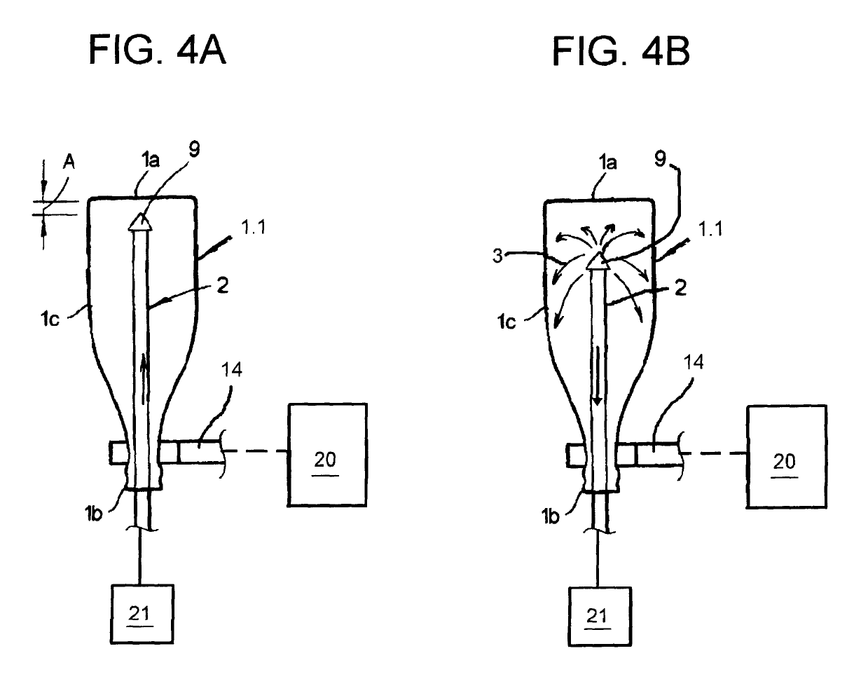

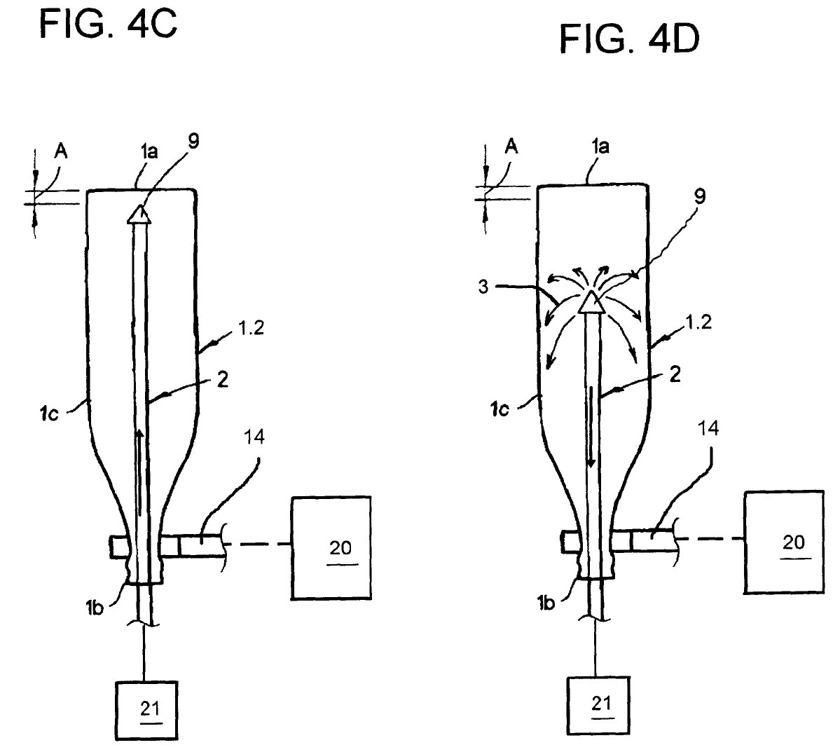

FIG. 4A shows one possible embodiment of the present application in use with a bottle 1.1 of a first size. After the bottle 1.1 is fed into the rotary treatment machine, the first step in treating the bottle 1.1 is insert the spray element 2 into the bottle mouth 1b. In FIG. 4A, the spray element 2 is inserted into the bottle 1.1 by the relative movement of the spray element 2 and the bottle 1 by means of motors 20 and 21. The nozzle 9 is a predetermined distance A away from the bottom 1a of the bottle 1.1.

The next step of the treatment process is seen in FIG. 4B. Once the nozzle 9 is the predetermined distance A away from the bottom 1a of the bottle 1.1, the spray element 2 is removed from the bottle 1.1 by relative movement of the spray element 2 and the bottle 1.1. As the spray element 2 is being removed from the bottle 1.1, the cleaning and/or disinfecting agent 3 is discharged from the nozzle 9 to promote optimal cleaning or disinfecting. When the nozzle 9 is in the vicinity of the mouth 1b of the bottle 1.1, the discharge of cleaning/disinfecting agent 3 is stopped. To complete the process, the spray element 2 is reinserted into the bottle 1.1 until a predetermined distance A between the nozzle 9 and the bottle 1a of the container 1.1 is reached. Once the predetermined distance A is reached, the spray element 2 is again removed and a second cleaning and/or disinfecting element 3′ is discharged from the nozzle 9. After the bottle 1.1 is treated with the second cleaning and/or disinfecting element 3′, the bottle 1.1 is then transferred out of the rotary treatment machine.

FIG. 4C shows the possible embodiment of the present application of FIG. 4A in use with a bottle 1.2 of a second size, which is larger than the bottle 1.1. The process is similar to that seen in FIGS. 4A and 4B. After the bottle 1.2 is fed into the rotary treatment machine, the first step in treating the bottle 1.2 is to insert the spray element 2 into the bottle mouth 1b. In FIG. 4C, the spray element 2 is inserted into the bottle 1.2 by the relative movement of the spray element 2 and the bottle 1 by means of motors 20 and 21. The nozzle 9 is a predetermined distance A away from the bottom 1a of the bottle 1.2.

The next step of the treatment process is seen in FIG. 4D. Once the nozzle 9 is the predetermined distance A away from the bottom 1a of the bottle 1.2, the spray element 2 is removed from the bottle 1.2 by relative movement of the spray element 2 and the bottle 1.2. As the spray element 2 is being removed, the cleaning and/or disinfecting agent 3 is discharged from the nozzle 9 to promote optimal cleaning or disinfecting. When the nozzle 9 is in the vicinity of the mouth 1b of the bottle 1.1, the discharge of cleaning/disinfecting agent 3 is stopped. To complete the process, the spray element 2 is reinserted into the bottle 1.2 until a predetermined distance A between the nozzle 9 and the bottle 1a of the container 1.2 is reached. Once the predetermined distance A is reached, the spray element 2 is again removed and a second cleaning and/or disinfecting element 3′ is discharged from the nozzle 9. After the bottle 1.2 is treated with the second cleaning and/or disinfecting element 3′, the bottle 1.2 is then transferred out of the rotary treatment machine.

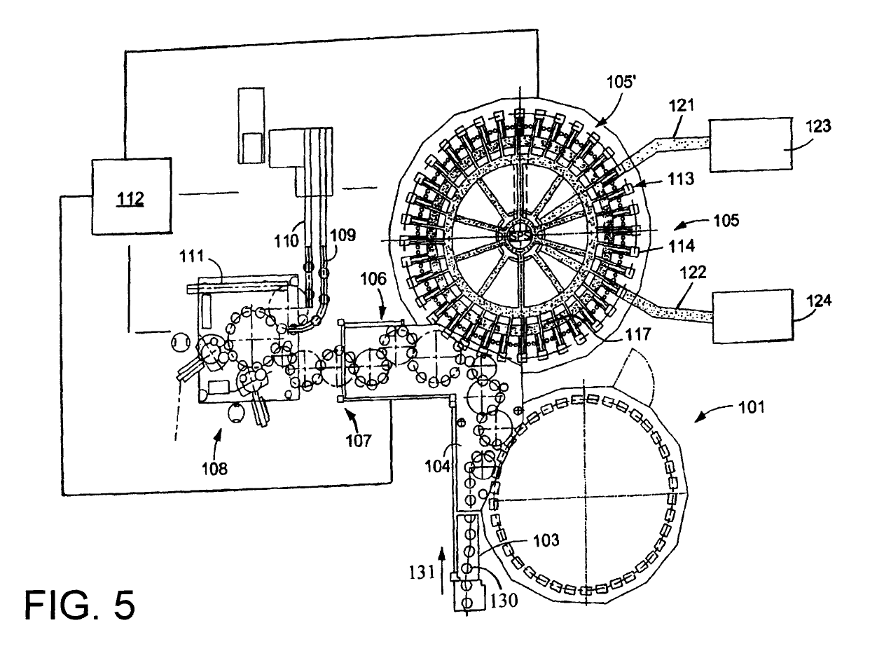

FIG. 5 shows schematically the main components of one possible embodiment example of a system for filling containers, specifically, a beverage bottling plant for filling bottles 130 with at least one liquid beverage, in accordance with at least one possible embodiment, in which system or plant could possibly be utilized at least one aspect, or several aspects, of the embodiments disclosed herein.

FIG. 5 shows a rinsing arrangement or rinsing station 101, to which the containers, namely bottles 130, are fed in the direction of travel as indicated by the arrow 131, by a first conveyer arrangement 103, which can be a linear conveyor or a combination of a linear conveyor and a starwheel. Downstream of the rinsing arrangement or rinsing station 101, in the direction of travel as indicated by the arrow 131, the rinsed bottles 130 are transported to a beverage filling machine 105 by a second conveyer arrangement 104 that is formed, for example, by one or more starwheels that introduce bottles 130 into the beverage filling machine 105.

The beverage filling machine 105 shown is of a revolving or rotary design, with a rotor 105′, which revolves around a central, vertical machine axis. The rotor 105′ is designed to receive and hold the bottles 130 for filling at a plurality of filling positions 113 located about the periphery of the rotor 105′. At each of the filling positions 103 is located a filling arrangement 114 having at least one filling device, element, apparatus, or valve. The filling arrangements 114 are designed to introduce a predetermined volume or amount of liquid beverage into the interior of the bottles 130 to a predetermined or desired level.

The filling arrangements 114 receive the liquid beverage material from a toroidal or annular vessel 117, in which a supply of liquid beverage material is stored under pressure by a gas. The toroidal vessel 117 is a component, for example, of the revolving rotor 105′. The toroidal vessel 117 can be connected by means of a rotary coupling or a coupling that permits rotation. The toroidal vessel 117 is also connected to at least one external reservoir or supply of liquid beverage material by a conduit or supply line. In the embodiment shown in FIG. 5, there are two external supply reservoirs 123 and 124, each of which is configured to store either the same liquid beverage product or different products. These reservoirs 123, 124 are connected to the toroidal or annular vessel 117 by corresponding supply lines, conduits, or arrangements 121 and 122. The external supply reservoirs 123, 124 could be in the form of simple storage tanks, or in the form of liquid beverage product mixers, in at least one possible embodiment.

As well as the more typical filling machines having one toroidal vessel, it is possible that in at least one possible embodiment there could be a second toroidal or annular vessel which contains a second product. In this case, each filling arrangement 114 could be connected by separate connections to each of the two toroidal vessels and have two individually-controllable fluid or control valves, so that in each bottle 130, the first product or the second product can be filled by means of an appropriate control of the filling product or fluid valves.

Downstream of the beverage filling machine 105, in the direction of travel of the bottles 130, there can be a beverage bottle closing arrangement or closing station 106 which closes or caps the bottles 130. The beverage bottle closing arrangement or closing station 106 can be connected by a third conveyer arrangement 107 to a beverage bottle labeling arrangement or labeling station 108. The third conveyor arrangement may be formed, for example, by a plurality of starwheels, or may also include a linear conveyor device.

In the illustrated embodiment, the beverage bottle labeling arrangement or labeling station 108 has at least one labeling unit, device, or module, for applying labels to bottles 130. In the embodiment shown, the labeling arrangement 108 is connected by a starwheel conveyer structure to three output conveyer arrangements: a first output conveyer arrangement 109, a second output conveyer arrangement 110, and a third output conveyer arrangement 111, all of which convey filled, closed, and labeled bottles 130 to different locations.

The first output conveyer arrangement 109, in the embodiment shown, is designed to convey bottles 130 that are filled with a first type of liquid beverage supplied by, for example, the supply reservoir 123. The second output conveyer arrangement 110, in the embodiment shown, is designed to convey bottles 130 that are filled with a second type of liquid beverage supplied by, for example, the supply reservoir 124. The third output conveyer arrangement 111, in the embodiment shown, is designed to convey incorrectly labeled bottles 130. To further explain, the labeling arrangement 108 can comprise at least one beverage bottle inspection or monitoring device that inspects or monitors the location of labels on the bottles 130 to determine if the labels have been correctly placed or aligned on the bottles 130. The third output conveyer arrangement 111 removes any bottles 130 which have been incorrectly labeled as determined by the inspecting device.

The beverage bottling plant can be controlled by a central control arrangement 112, which could be, for example, computerized control system that monitors and controls the operation of the various stations and mechanisms of the beverage bottling plant.

The objects of the present application are a method and an apparatus for the treatment of containers 1, in one possible embodiment for the upside-down cleaning of bottles 1. At least one sprayer element 2 thereby sprays the interior of the container with a disinfecting/cleaning agent 3, 3′. The invention teaches that the sprayer element 2 is inserted into the interior of the container, in one possible embodiment into the vicinity of the bottom 1a of the container.

One feature or aspect of an embodiment is believed at the time of the filing of this patent application to possibly reside broadly in a method for the treatment of containers 1, in one possible embodiment for the upside-down cleaning of bottles 1, in which at least one sprayer element 2 sprays the interior of the container with a disinfecting/cleaning agent 3, 3′, wherein the sprayer element 2 is inserted into the interior of the container, in one possible embodiment into the vicinity of the bottom 1a of the container.

Another feature or aspect of an embodiment is believed at the time of the filing of this patent application to possibly reside broadly in the method, wherein the sprayer element 2 and the container 1 vary their relative distance A from each other.

Yet another feature or aspect of an embodiment is believed at the time of the filing of this patent application to possibly reside broadly in the method, wherein the sprayer element 2 is inserted into and removed from the interior of the container a plurality of times during a cleaning/disinfecting routine.

Still another feature or aspect of an embodiment is believed at the time of the filing of this patent application to possibly reside broadly in the method, wherein during one cleaning/disinfecting routine the sprayer element 2 is supplied with a plurality of different disinfecting/cleaning agents 3, 3′, for example water 3 and air 3′ and/or the sprayer element 2 emits a type of radiation during the treatment.

A further feature or aspect of an embodiment is believed at the time of the filing of this patent application to possibly reside broadly in the method, wherein the sprayer element 2 is first inserted into the container 1, sprays it with a first disinfecting/cleaning agent 3 and is then removed and then re-inserted into the interior of the container, which is sprayed with a second disinfecting/cleaning agent 3′.

Another feature or aspect of an embodiment is believed at the time of the filing of this patent application to possibly reside broadly in the method, wherein the sprayer element 2 is moved relative to the container 1 during the cleaning/disinfecting routine.

Yet another feature or aspect of an embodiment is believed at the time of the filing of this patent application to possibly reside broadly in an apparatus for the treatment of containers 1, in one possible embodiment for the upside down cleaning of bottles 1, in one possible embodiment for the performance of the method according to the present application, with at least one sprayer element 2 which sprays the interior of the container with a disinfecting/cleaning agent 3, 3′, wherein the sprayer element 2 and/or the container 1 can be moved to vary their relative distance A from each other.

Still another feature or aspect of an embodiment is believed at the time of the filing of this patent application to possibly reside broadly in the apparatus, wherein the sprayer element 2 is realized so that it can be inserted into the interior of the container in one possible embodiment into the vicinity of the bottom 1a of the container.

A further feature or aspect of an embodiment is believed at the time of the filing of this patent application to possibly reside broadly in the apparatus, wherein the sprayer element 2 is fastened to a variable-height actuator device 4, 5, 6, 7.

Another feature or aspect of an embodiment is believed at the time of the filing of this patent application to possibly reside broadly in the apparatus, wherein the variable-height actuator device 4, 5, 6, 7 comprises essentially a base section 4, 5 and a control section 6, 7, whereby the base section 4, 5 supports the sprayer element 2 and the control section 6, 7 positions the base section 4, 5.

Yet another feature or aspect of an embodiment is believed at the time of the filing of this patent application to possibly reside broadly in the apparatus, wherein the sprayer element 2 is realized in the form of a sprayer tube 2 with an optional wrap-around nozzle 9 on the head.

Still another feature or aspect of an embodiment is believed at the time of the filing of this patent application to possibly reside broadly in the apparatus, wherein an inlet conveyor 11 an outlet conveyor 13 and a treatment conveyor 12 are provided for the containers 1, whereby the containers 1 are transferred into their treatment position and back again in the inlet conveyor 11 and/or in the outlet conveyor 13 and/or before or after said conveyors.

A further feature or aspect of an embodiment is believed at the time of the filing of this patent application to possibly reside broadly in a method for the treatment of containers 1, in one possible embodiment for the upside-down cleaning of bottles 1, in which at least one sprayer element 2 sprays the interior of the container with a disinfecting/cleaning agent 3, 3′, and in which the sprayer element 2 is inserted into the interior of the container, in one possible embodiment into the vicinity of the bottom 1a of the container, wherein

the sprayer element 2 is first inserted into the container 1, supplied with a first disinfecting/cleaning agent 3 and then extracted, and

then the sprayer element 2 is again inserted into the interior of the container and supplied with a second disinfecting/cleaning agent 3′, whereby

additional types of bottles can also be processed.

One feature or aspect of an embodiment is believed at the time of the filing of this patent application to possibly reside broadly in the method, wherein the sprayer element 2 and the container 1 vary their relative distance A from each other.

Another feature or aspect of an embodiment is believed at the time of the filing of this patent application to possibly reside broadly in the method, wherein the sprayer element 2 is inserted into and removed from the interior of the container a plurality of times during a cleaning/disinfecting routine.

Yet another feature or aspect of an embodiment is believed at the time of the filing of this patent application to possibly reside broadly in the method, wherein during one cleaning/disinfecting routine the sprayer element 2 is supplied with a plurality of different disinfecting/cleaning agents 3, 3′, for example water 3 and air 3′.

Still another feature or aspect of an embodiment is believed at the time of the filing of this patent application to possibly reside broadly in the method, wherein the sprayer element 2 is moved relative to the container 1 during the cleaning/disinfecting routine.

A further feature or aspect of an embodiment is believed at the time of the filing of this patent application to possibly reside broadly in an apparatus for the treatment of containers 1, in one possible embodiment for the upside-down cleaning of bottles 1, in one possible embodiment for the performance of the method according to the present application, with at least one sprayer element 2 which sprays the interior of the container with a disinfecting/cleaning agent 3, 3′, whereby the sprayer element 2 and/or the container 1 can be moved to vary their relative distance A from each other and for that purpose the sprayer element 2 is fastened to a vertically adjustable actuator apparatus 4, 5, 6, 7, wherein

the variable-height actuator apparatus 4, 5, 6, 7 comprises essentially a base section 4, 5 and a control section 6, 7, whereby

the base section 4, 5 supports the sprayer element 2 and the control section 6, 7 positions the base section, and whereby

the control section 6, 7 also has a linear actuator 6.

Another feature or aspect of an embodiment is believed at the time of the filing of this patent application to possibly reside broadly in the apparatus, wherein the sprayer element 2 is realized so that it can be inserted into the interior of the container in one possible embodiment into the vicinity of the bottom 1a of the container.

Yet another feature or aspect of an embodiment is believed at the time of the filing of this patent application to possibly reside broadly in the apparatus, wherein the sprayer element 2 is realized in the form of a sprayer tube 2 with an optional wrap-around nozzle 9 on the head.

Still another feature or aspect of an embodiment is believed at the time of the filing of this patent application to possibly reside broadly in the apparatus, wherein an inlet conveyor 11 an outlet conveyor 13 and a treatment conveyor 12 are provided for the containers 1, whereby the containers 1 are transferred into their treatment position and back again in the inlet conveyor 11 and/or in the outlet conveyor 13 and/or before or after said conveyors.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

All of the patents, patent applications or patent publications, which were cited in the International Search Report dated Feb. 13, 2008, and/or cited elsewhere are hereby incorporated by reference as if set forth in their entirety herein as follows: EP 0 381 841 having the following English translation of the German title, “METHOD AND DEVICE FOR CLEANING AND STERILIZING CONTAINERS,” published on Aug. 16, 1990; FR 2,873,310, having the following English translation of the French title “MULTIPLE STATION MACHINE FOR CLEANING A CONTAINER BY SCOURING WITH A COMPRESSED GAS PERIPHERAL JET,” published on Jan. 1, 2006; NL 278 223, having the Dutch title “INJECTEUR VOOR SPOELMACHINES VAN HOUDERS EN EEN SPOELMACHINE VOORZIEN VAN EEN DERGELIJKE INJECTEUR,” published on Nov. 10, 1964; and DE 299 03 939, having the German title “VORRICHTUNG ZUR ÜBERKOPF-BEHANDLUNG VON FLASCHEN,” published on Apr. 16, 2000.

All of the patents, patent applications or patent publications, which were cited in the German Office Action dated Dec. 5, 2007, and/or cited elsewhere are hereby incorporated by reference as if set forth in their entirety herein as follows: DE 41 33 448, having the following English translation of the German title, “WASHING EQUIPMENT FOR REUSABLE BOTTLES—HAS VERTICALLY MOVING NEEDLES TO SPRAY PRESSURISED WATER AGAINST SURFACES OF INVERTED POLYETHYLENE TEREPHTHALATE BOTTLES,” published on Apr. 15, 1993; and DE 42 29 580, having the German title “VERFAHREN UND VORRICHTUNG ZUR ÜBERKOPF-BEHANDLUNG VON FLASCHEN,” published on Mar. 10, 1994.

U.S. patent application Ser. No. 12/389,937, filed on Feb. 20, 2009, having inventors Bernd MOLITOR and Klaus JENDRICHOWSKI, Attorney Docket No. NHL-HOL-217, and title “METHOD OF CLEANING BOTTLES IN A BOTTLE CLEANING MACHINE, AND AN ARRANGEMENT FOR PERFORMING THE METHOD, AND A BOTTLE CLEANING MACHINE,” and its corresponding Federal Republic of Germany Patent Application No. 10 2006 039 599.9, filed on Aug. 23, 2006, and International Patent Application No. PCT/EP2007/007242, filed on Aug. 16, 2007, having WIPO Publication No. WO 2008/022740 and inventors Bernd MOLITOR and Klaus JENDRICHOWSKI are hereby incorporated by reference as if set forth in their entirety herein.

The patents, patent applications, and patent publication listed above in the preceding three paragraphs, beginning with the phrase: “All of the patents, patent applications or patent publications, which were cited in the International Search Report . . . ” and ending with the phrase: “ . . . and inventors Bernd MOLITOR and Klaus JENDRICHOWSKI are hereby incorporated by reference as if set forth in their entirety herein” are herein incorporated by reference as if set forth in their entirety. The purpose of incorporating U.S. patents, Foreign patents, publications, etc. is solely to provide additional information relating to technical features of one or more embodiments, which information may not be completely disclosed in the wording in the pages of this application. Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

The corresponding foreign and international patent publication applications, namely, Federal Republic of Germany Patent Application No. 10 2006 044 904.5, filed on Sep. 22, 2006, having inventors Klaus BAUMGARTNER, Steffen KAPPEL, and Andreas FAHLDIECK, and DE-OS 10 2006 044 904.5 and DE-PS 10 2006 044 904.5, and International Application No. PCT/EP2007/007475, filed on Aug. 25, 2007, having WIPO Publication No. WO 2008/034512 and inventors Klaus BAUMGARTNER, Steffen KAPPEL, and Andreas FAHLDIECK, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

The purpose of incorporating the Foreign equivalent patent application PCT/EP2007/007475 and German Patent Application 10 2006 044 904.5 is solely for the purpose of providing a basis of correction of any wording in the pages of the present application, which may have been mistranslated or misinterpreted by the translator. Words relating to opinions and judgments of the author and not directly relating to the technical details of the description of the embodiments therein are not to be incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned word in this sentence, when not used to describe technical features of one or more embodiments, are not generally considered to be incorporated by reference herein.

Statements made in the original foreign patent applications PCT/EP2007/007475 and DE 10 2006 044 904.5 from which this patent application claims priority which do not have to do with the correction of the translation in this patent application are not to be included in this patent application in the incorporation by reference.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.
   Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

1. A method of cleaning beverage bottles in a beverage bottling plant; said method comprising the steps of:
(A) inverting a first beverage bottle and positioning the base of said first beverage bottle above the mouth of said first beverage bottle, said first beverage bottle comprising a first physical configuration;
(B) feeding said inverted first beverage bottle into a beverage bottle cleaning machine;
(C) inserting a sprayer element into said mouth of said inverted first beverage bottle through relative movement of said sprayer element and said inverted first beverage bottle;
(D) moving a discharge element on said sprayer element to a first predetermined distance away from said base of said inverted first beverage bottle, which first predetermined distance is sufficient to maximize cleaning of said inverted first beverage bottle while minimizing the possibility of damage to said base of said inverted first beverage bottle;
(E) at least substantially withdrawing said sprayer element from said inverted first beverage bottle while substantially simultaneously discharging a first cleaning agent from said discharge element with a sufficient quantity of said first cleaning agent and at a sufficient pressure to maximize cleaning of said inverted first beverage bottle;
(F) stopping the discharge of said first cleaning agent from said discharge element upon said discharge element being disposed a second predetermined distance away from said base of said inverted first beverage bottle, said second predetermined distance being substantially different than said first predetermined distance;
(G) moving said sprayer element into said inverted first beverage bottle through relative movement of said sprayer element and said inverted first beverage bottle;
(H) moving said discharge element on said sprayer element to a third predetermined distance away from said base of said inverted first beverage bottle, which third predetermined distance is sufficient to maximize cleaning of said inverted first beverage bottle while minimizing the possibility of damage to said base of said inverted first beverage bottle;
(I) at least substantially withdrawing said sprayer element from said inverted first beverage bottle while substantially simultaneously discharging a second cleaning agent from said discharge element with a sufficient quantity of said second cleaning agent and at a sufficient pressure to maximize cleaning of said inverted first beverage bottle;
(J) stopping the discharge of said second cleaning agent from said discharge element upon said discharge element being disposed a fourth predetermined distance away from said base of said inverted first beverage bottle, said fourth predetermined distance being substantially different than said third predetermined distance;
(K) moving said inverted first beverage bottle out of said beverage bottle cleaning machine;
(L) inverting said inverted first beverage bottle and positioning the mouth of said first beverage bottle above the base of said first beverage bottle;
(M) inverting a second beverage bottle and positioning the base of said second beverage bottle above the mouth of said second beverage bottle, said second beverage bottle comprising a second physical configuration, which second physical configuration is different than said first physical configuration;
(N) feeding said inverted second beverage bottle into said beverage bottle cleaning machine;
(O) inserting said sprayer element into said mouth of said inverted second beverage bottle through relative movement of said sprayer element and said inverted second beverage bottle;
(P) moving said discharge element on said sprayer element to a fifth predetermined distance away from said base of said inverted second beverage bottle, which fifth predetermined distance is sufficient to maximize cleaning of said inverted second beverage bottle while minimizing the possibility of damage to said base of said inverted second beverage bottle;
(Q) at least substantially withdrawing said sprayer element from said inverted second beverage bottle while substantially simultaneously discharging said first cleaning agent from said discharge element with a sufficient quantity of said first cleaning agent and at a sufficient pressure to maximize cleaning of said inverted second beverage bottle;
(R) stopping the discharge of said first cleaning agent from said discharge element upon said discharge element being disposed a sixth predetermined distance away from said base of said inverted second beverage bottle, said sixth predetermined distance being substantially different than said fifth predetermined distance;
(S) moving said sprayer element into said inverted second beverage bottle through relative movement of said sprayer element and said inverted second beverage bottle;
(T) moving said discharge element on said sprayer element to a seventh predetermined distance away from said base of said inverted second beverage bottle, which seventh predetermined distance is sufficient to maximize cleaning of said inverted second beverage bottle while minimizing the possibility of damage to said base of said inverted second beverage bottle;
(U) at least substantially withdrawing said sprayer element from said inverted second beverage bottle while substantially simultaneously discharging said second cleaning agent from said discharge element with a sufficient quantity of said second cleaning agent and at a sufficient pressure to maximize cleaning of said inverted second beverage bottle;
(V) stopping the discharge of said second cleaning agent from said discharge element upon said discharge element being disposed an eighth predetermined distance away from said base of said inverted second beverage bottle, said eighth predetermined distance being substantially different than said seventh predetermined distance;
(W) moving said inverted second beverage bottle out of said beverage bottle cleaning machine;
(X) inverting said inverted second beverage bottle and positioning the mouth of said second beverage bottle above the base of said second beverage bottle;
(Y) continuing steps A-L with beverage bottles of said first physical configuration; and
(Z) continuing steps M-X with beverage bottles of said second physical configuration.

2. Means for performing the method of cleaning beverage bottles in a beverage bottling plant according to claim 1; said means comprising:
a first means for inverting a first beverage bottle and positioning a base of the first beverage bottle above a mouth of the first beverage bottle, the first beverage bottle comprising a first physical configuration;
means for feeding an inverted first beverage bottle into a beverage bottle cleaning machine;
means for inserting a sprayer element into a mouth of an inverted first beverage bottle through relative movement of said sprayer element and the inverted first beverage bottle;
means for moving a discharge element on said sprayer element to a first predetermined distance away from a base of an inverted first beverage bottle, which first predetermined distance is sufficient to maximize cleaning of an inverted first beverage bottle while minimizing the possibility of damage to the base of the inverted first beverage bottle;
means for at least substantially withdrawing said sprayer element from an inverted first beverage bottle while substantially simultaneously discharging a first cleaning agent from said discharge element with a sufficient quantity of the first cleaning agent and at a sufficient pressure to maximize cleaning of the inverted first beverage bottle;
means for stopping the discharge of a first cleaning agent from said discharge element upon said discharge element being disposed a second predetermined distance away from a base of an inverted first beverage bottle, the second predetermined distance being substantially different than the first predetermined distance;
said means for moving comprising means for moving said sprayer element into an inverted first beverage bottle through relative movement of said sprayer element and the inverted first beverage bottle;
said means for moving comprising means for moving said discharge element on said sprayer element to a third predetermined distance away from a base of an inverted first beverage bottle, which third predetermined distance is sufficient to maximize cleaning of the inverted first beverage bottle while minimizing the possibility of damage to the base of the inverted first beverage bottle;
said means for at least substantially withdrawing comprising means for at least substantially withdrawing said sprayer element from an inverted first beverage bottle while substantially simultaneously discharging a second cleaning agent from said discharge element with a sufficient quantity of the second cleaning agent and at a sufficient pressure to maximize cleaning of the inverted first beverage bottle;
said means for stopping comprising means for stopping the discharge of a second cleaning agent from said discharge element upon said discharge element being disposed a fourth predetermined distance away from a base of an inverted first beverage bottle, the fourth predetermined distance being substantially different than the third predetermined distance;
said means for moving comprising means for moving an inverted first beverage bottle out of said beverage bottle cleaning machine;
a second means for inverting an inverted first beverage bottle and positioning the mouth of the first beverage bottle above the base of the first beverage bottle;
said first means for inverting comprising means for inverting a second beverage bottle and positioning a base of the second beverage bottle above a mouth of the second beverage bottle, the second beverage bottle comprising a second physical configuration, which second physical configuration is different than the first physical configuration;
said means for feeding comprising means for feeding an inverted second beverage bottle into said beverage bottle cleaning machine;
said means for inserting comprising means for inserting said sprayer element into a mouth of an inverted second beverage bottle through relative movement of said sprayer element and the inverted second beverage bottle;
said means for moving comprising means for moving said discharge element on said sprayer element to a fifth predetermined distance away from a base of an inverted second beverage bottle, which fifth predetermined distance is sufficient to maximize cleaning of the inverted second beverage bottle while minimizing the possibility of damage to the base of the inverted second beverage bottle;
said means for at least substantially withdrawing comprising means for at least substantially withdrawing said sprayer element from an inverted second beverage bottle while substantially simultaneously discharging a first cleaning agent from said discharge element with a sufficient quantity of the first cleaning agent and at a sufficient pressure to maximize cleaning of the inverted second beverage bottle;
said means for stopping comprising means for stopping the discharge of a first cleaning agent from said discharge element upon said discharge element being disposed a sixth predetermined distance away from a base of an inverted second beverage bottle, the sixth predetermined distance being substantially different than the fifth predetermined distance;
said means for moving comprising means for moving said sprayer element into an inverted second beverage bottle through relative movement of said sprayer element and the inverted second beverage bottle;
said means for moving comprising means for moving said discharge element on said sprayer element to a seventh predetermined distance away from a base of an inverted second beverage bottle, which seventh predetermined distance is sufficient to maximize cleaning of the inverted second beverage bottle while minimizing the possibility of damage to the base of the inverted second beverage bottle;
said means for at least substantially withdrawing comprising means for at least substantially withdrawing said sprayer element from an inverted second beverage bottle while substantially simultaneously discharging a second cleaning agent from said discharge element with a sufficient quantity of the second cleaning agent and at a sufficient pressure to maximize cleaning of the inverted second beverage bottle;
said means for stopping comprising means for stopping the discharge of a second cleaning agent from said discharge element upon said discharge element being disposed an eighth predetermined distance away from a base of an inverted second beverage bottle, the eighth predetermined distance being substantially different than the seventh predetermined distance;
said means for moving comprising means for moving an inverted second beverage bottle out of said beverage bottle cleaning machine;
said second means for inverting comprising means for inverting an inverted second beverage bottle and positioning the mouth of the second beverage bottle above the base of the second beverage bottle;
means for continuing the method with beverage bottles of the first physical configuration; and
said means for continuing comprising means for continuing the method with beverage bottles of the second physical configuration.

3. A beverage bottle cleaning arrangement for performing the method of cleaning beverage bottles in a beverage bottling plant according to claim 1; said beverage bottle cleaning arrangement comprising:
a first inverting arrangement being configured to invert a first beverage bottle and position a base of the first beverage bottle above a mouth of the first beverage bottle, the first beverage bottle comprising a first physical configuration;
a feeding arrangement being configured to feed an inverted first beverage bottle into a beverage bottle cleaning machine;
an inserting arrangement being configured to insert a sprayer element into a mouth of an inverted first beverage bottle through relative movement of said sprayer element and the inverted first beverage bottle;
a moving arrangement being configured to move a discharge element on said sprayer element to a first predetermined distance away from a base of an inverted first beverage bottle, which first predetermined distance is sufficient to maximize cleaning of an inverted first beverage bottle while minimizing the possibility of damage to the base of the inverted first beverage bottle;
a withdrawing arrangement being configured to at least substantially withdraw said sprayer element from an inverted first beverage bottle while being further configured to substantially simultaneously discharge a first cleaning agent from said discharge element with a sufficient quantity of the first cleaning agent and at a sufficient pressure to maximize cleaning of the inverted first beverage bottle;
a stopping arrangement being configured to stop the discharge of a first cleaning agent from said discharge element upon said discharge element being disposed a second predetermined distance away from a base of an inverted first beverage bottle, the second predetermined distance being substantially different than the first predetermined distance;
said moving arrangement is further configured to move said sprayer element into an inverted first beverage bottle through relative movement of said sprayer element and the inverted first beverage bottle;
said moving arrangement is further configured to move said discharge element on said sprayer element to a third predetermined distance away from a base of an inverted first beverage bottle, which third predetermined distance is sufficient to maximize cleaning of the inverted first beverage bottle while minimizing the possibility of damage to the base of the inverted first beverage bottle;
said withdrawing arrangement is further configured to at least substantially withdraw said sprayer element from an inverted first beverage bottle while being further configured to substantially simultaneously discharge a second cleaning agent from said discharge element with a sufficient quantity of the second cleaning agent and at a sufficient pressure to maximize cleaning of the inverted first beverage bottle;
said stopping arrangement is further configured to stop the discharge of a second cleaning agent from said discharge element upon said discharge element being disposed a fourth predetermined distance away from a base of an inverted first beverage bottle, the fourth predetermined distance being substantially different than the third predetermined distance;
said moving arrangement is further configured to move an inverted first beverage bottle out of said beverage bottle cleaning machine;
a second inverting arrangement being configured to invert an inverted first beverage bottle and position the mouth of the first beverage bottle above the base of the first beverage bottle;
said first inverting arrangement is further configured to invert a second beverage bottle and position a base of the second beverage bottle above a mouth of the second beverage bottle, the second beverage bottle comprising a second physical configuration, which second physical configuration is different than the first physical configuration;
said feeding arrangement is further configured to feed an inverted second beverage bottle into said beverage bottle cleaning machine;
said inserting arrangement is further configured to insert said sprayer element into a mouth of an inverted second beverage bottle through relative movement of said sprayer element and the inverted second beverage bottle;
said moving arrangement is further configured to move said discharge element on said sprayer element to a fifth predetermined distance away from a base of an inverted second beverage bottle, which fifth predetermined distance is sufficient to maximize cleaning of the inverted second beverage bottle while minimizing the possibility of damage to the base of the inverted second beverage bottle;
said withdrawing arrangement is further configured to at least substantially withdraw said sprayer element from an inverted second beverage bottle while being further configured to substantially simultaneously discharge a first cleaning agent from said discharge element with a sufficient quantity of the first cleaning agent and at a sufficient pressure to maximize cleaning of the inverted second beverage bottle;
said stopping arrangement is further configured to stop the discharge of a first cleaning agent from said discharge element upon said discharge element being disposed a sixth predetermined distance away from a base of an inverted second beverage bottle, the sixth predetermined distance being substantially different than the fifth predetermined distance;
said moving arrangement is further configured to move said sprayer element into an inverted second beverage bottle through relative movement of said sprayer element and the inverted second beverage bottle;
said moving arrangement is further configured to move said discharge element on said sprayer element to a seventh predetermined distance away from a base of an inverted second beverage bottle, which seventh predetermined distance is sufficient to maximize cleaning of the inverted second beverage bottle while minimizing the possibility of damage to the base of the inverted second beverage bottle;
said withdrawing arrangement is further configured to at least substantially withdraw said sprayer element from an inverted second beverage bottle while being further configured to substantially simultaneously discharge a second cleaning agent from said discharge element with a sufficient quantity of the second cleaning agent and at a sufficient pressure to maximize cleaning of the inverted second beverage bottle;
said stopping arrangement is further configured to stop the discharge of a second cleaning agent from said discharge element upon said discharge element being disposed an eighth predetermined distance away from a base of an inverted second beverage bottle, the eighth predetermined distance being substantially different than the seventh predetermined distance;
said moving arrangement is further configured to move an inverted second beverage bottle out of said beverage bottle cleaning machine;
said second inverting arrangement is further configured to invert an inverted second beverage bottle and positioning the mouth of the second beverage bottle above the base of the second beverage bottle;
a continuing arrangement being configured to continue the method with beverage bottles of the first physical configuration; and
said continuing arrangement is further configured to continue the method with beverage bottles of the second physical configuration.

4. The method of cleaning beverage bottles in a beverage bottling plant according to claim 1, wherein:
said method further comprises repeating steps C-J and steps O-V a plurality of times;
said first cleaning agent comprises water;
said second cleaning agent comprises air;
said sprayer element is configured to move relative to said inverted first beverage bottle; and
said sprayer element is configured to move relative to said inverted second beverage bottle.

5. The method of cleaning beverage bottles in a beverage bottling plant according to claim 4 including a substantially vertically adjustable actuator apparatus, a linear actuator, an inlet conveyor, an outlet conveyor, and a treatment conveyor, wherein:
said sprayer element is operatively connected to said substantially vertically adjustable actuator apparatus comprising a base and a control section;
said control section is configured to adjust the substantially vertical height of said base;
said control section is operatively connected to said linear actuator, which linear actuator is configured to adjust the substantially vertical height of said control section;
said sprayer element is configured to be moved, inside said inverted first a beverage bottle, to a portion of said inverted first beverage bottle substantially immediately adjacent said base of said inverted first beverage bottle;
said sprayer element is configured to be moved, inside said inverted second a beverage bottle, to a portion of said inverted second beverage bottle substantially immediately adjacent said base of said inverted second a beverage bottle;
said discharge element is configured to spray in all directions around said discharge element;
said step of inverting a first beverage bottle and positioning the base of said first beverage bottle above the mouth of said first beverage bottle further comprises inserting said first beverage bottle and positioning said base of said first beverage bottle above said mouth of said first a beverage bottle in said inlet conveyor;
said step of inverting a second beverage bottle and positioning the base of said second beverage bottle above the mouth of said second beverage bottle further comprises inverting said second beverage bottle and positioning said base of said second beverage bottle above said mouth of said second beverage bottle in said inlet conveyor;
said step of inverting said inverted first beverage bottle and positioning the mouth of said first beverage bottle above the base of said first beverage bottle further comprises inverting said inverted first beverage bottle and positioning said mouth of said first a beverage bottle above said base of said first beverage bottle in said outlet conveyor;
said step of inverting said inverted second beverage bottle and positioning the mouth of said second a beverage bottle above the base of said second beverage bottle further comprises inverting said inverted second beverage bottle and positioning the mouth of said second a beverage bottle above the base of said second beverage bottle in said outlet conveyor; and
said method further comprises:
moving said inverted first beverage bottle along said treatment conveyor between said inlet conveyor and said outlet conveyor; and
moving said inverted second beverage bottle along said treatment conveyor between said inlet conveyor and said outlet conveyor.

6. A method of cleaning containers in a container filling plant; said method comprising the steps of:
(A) positioning a base of a first container above a mouth of said first container, said first container comprising a first physical configuration;
(B) inserting a sprayer element into said mouth of said inverted first container through relative movement of said sprayer element and said inverted first container;
(C) moving said sprayer element to a first substantially predetermined distance away from said base of said inverted first container;
(D) discharging a first cleaning agent from said sprayer element;
(E) moving said sprayer element away from said base of said inverted first container;
(F) stopping the discharge of said first cleaning agent from said sprayer element upon said sprayer element being disposed a second substantially predetermined distance away from said base of said inverted first container, said second substantially predetermined distance being substantially different from said first substantially predetermined distance;
(G) moving said sprayer element toward said base of said inverted first container through relative movement of said sprayer element and said inverted first container;
(H) moving said sprayer element to a third substantially predetermined distance away from said base of said inverted first container;
(I) discharging a second cleaning agent from said sprayer element;
(J) moving said sprayer element away from said base of said inverted first container;
(K) stopping the discharge of said second cleaning agent from said sprayer element upon said sprayer element being disposed a fourth substantially predetermined distance away from said base of said inverted first container, said fourth substantially predetermined distance being substantially different from said third substantially predetermined distance;
(L) positioning a base of a second container above a mouth of said second container, said second container comprising a second physical configuration, which second physical configuration is different than said first physical configuration;
(M) inserting said sprayer element into said mouth of said inverted second container through relative movement of said sprayer element and said inverted second container;
(N) moving said sprayer element to a fifth substantially predetermined distance away from said base of said inverted second container;
(O) discharging said first cleaning agent from said sprayer element;
(P) moving said sprayer element away from said base of said inverted second container;
(Q) stopping the discharge of said first cleaning agent from said sprayer element upon said sprayer element being disposed a sixth substantially predetermined distance away from said base of said inverted second container, said sixth substantially predetermined distance being substantially different from said fifth substantially predetermined distance;
(R) moving said sprayer element toward said base of said inverted second container through relative movement of said sprayer element and said inverted second container;
(S) moving said sprayer element to a seventh substantially predetermined distance away from said base of said inverted second container;
(T) discharging said second cleaning agent from said sprayer element;
(U) moving said sprayer element away from said base of said inverted second container;
(V) stopping the discharge of said second cleaning agent from said sprayer element upon said sprayer element being disposed an eighth substantially predetermined distance away from said base of said inverted second container, said eighth substantially predetermined distance being substantially different from said seventh substantially predetermined distance;
(W) continuing steps A-K with containers of said first physical configuration; and
(X) continuing steps L-V with containers of said second physical configuration.

7. Means for performing the method of cleaning containers in a container filling plant of claim 6; said means comprising:
means for positioning a base of a first container above the mouth of the first container, the first container comprising a first physical configuration;
means for inserting a sprayer element into a mouth of an inverted first container through relative movement of said sprayer element and the inverted first container;
means for moving said sprayer element to a first substantially predetermined distance away from a base of an inverted first container;
means for discharging a first cleaning agent from said sprayer element;
said means for moving comprising means for moving said sprayer element away from a base of an inverted first container;
means for stopping the discharge of a first cleaning agent from said sprayer element upon said sprayer element being disposed a second substantially predetermined distance away from a base of an inverted first container, the second substantially predetermined distance being substantially different from the first substantially predetermined distance;
said means for moving comprising means for moving said sprayer element toward a base of an inverted first container through relative movement of said sprayer element and the inverted first container;
said means for moving comprising means for moving said sprayer element to a third substantially predetermined distance away from a base of an inverted first container;
said means for discharging comprising means for discharging a second cleaning agent from said sprayer element;
said means for moving comprising means for moving said sprayer element away from a base of an inverted first container;
said means for stopping comprising means for stopping the discharge of a second cleaning agent from said sprayer element upon said sprayer element being disposed a fourth substantially predetermined distance away from a base of a inverted first container, the fourth substantially predetermined distance being substantially different from the third substantially predetermined distance;
said means for positioning comprising means for positioning a base of a second container above the mouth of the second container, the second container comprising a second physical configuration, which second physical configuration is different than the first physical configuration;
said means for inserting comprising means for inserting said sprayer element into a mouth of an inverted second container through relative movement of said sprayer element and the inverted second container;
said means for moving comprising means for moving said sprayer element to a fifth substantially predetermined distance away from a base of an inverted second container;
said means for discharging comprising means for discharging a first cleaning agent from said sprayer element;
said means for moving comprising means for moving said sprayer element away from a base of an inverted second container;
said means for stopping comprising means for stopping the discharge of a first cleaning agent from said sprayer element upon said sprayer element being disposed a sixth substantially predetermined distance away from a base of an inverted second container, the sixth substantially predetermined distance being substantially different from the fifth substantially predetermined distance;
said means for moving comprising means for moving said sprayer element toward a base of an inverted second container through relative movement of said sprayer element and the inverted second container;
said means for moving comprising means for moving said sprayer element to a seventh substantially predetermined distance away from a base of an inverted second container;
said means for discharging comprising means for discharging a second cleaning agent from said sprayer element;
said means for moving comprising means for moving said sprayer element away from a base of an inverted second container;
said means for stopping comprising means for stopping the discharge of a second cleaning agent from said sprayer element upon said sprayer element being disposed an eighth substantially predetermined distance away from a base of an inverted second container, the eighth substantially predetermined distance being substantially different from the seventh substantially predetermined distance;
means for continuing the method with containers of the first physical configuration; and
means for continuing the method with containers of the second physical configuration.

8. A container cleaning arrangement for performing the method of cleaning containers in a container filling plant of claim 6; said container cleaning arrangement comprising:
a positioning arrangement being configured to position a base of a first container above the mouth of the first container, the first container comprising a first physical configuration;
an inserting arrangement being configured to insert a sprayer element into a mouth of an inverted first container through relative movement of said sprayer element and the inverted first container;
a moving arrangement being configured to move said sprayer element to a first substantially predetermined distance away from a base of an inverted first container;
a discharging arrangement being configured to discharge a first cleaning agent from said sprayer element;
said moving arrangement is further configured to move said sprayer element away from a base of an inverted first container;
a stopping arrangement being configured to stop the discharge of a first cleaning agent from said sprayer element upon said sprayer element being disposed a second substantially predetermined distance away from a base of an inverted first container, the second substantially predetermined distance being substantially different from the first substantially predetermined distance;
said moving arrangement is further configured to move said sprayer element toward a base of an inverted first container through relative movement of said sprayer element and the inverted first container;
said moving arrangement is further configured to move said sprayer element to a third substantially predetermined distance away from a base of an inverted first container;
said discharging arrangement being further configured to discharge a second cleaning agent from said sprayer element;
said moving arrangement is further configured to move said sprayer element away from a base of an inverted first container;
said stopping arrangement is further configured to stop the discharge of a second cleaning agent from said sprayer element upon said sprayer element being disposed a fourth substantially predetermined distance away from a base of an inverted first container, the fourth substantially predetermined distance being substantially different from the third substantially predetermined distance;
said positioning arrangement is further configured to position a base of a second container above the mouth of the second container, the second container comprising a second physical configuration, which second physical configuration is different than the first physical configuration;
said inserting arrangement is further configured to insert said sprayer element into a mouth of an inverted second container through relative movement of said sprayer element and the inverted second container;
said moving arrangement is further configured to move said sprayer element to a fifth substantially predetermined distance away from a base of an inverted second container;
said discharging arrangement is further configured to discharge a first cleaning agent from said sprayer element;
said moving arrangement is further configured to move said sprayer element away from a base of an inverted second container;
said stopping arrangement is further configured to stop the discharge of a first cleaning agent from said sprayer element upon said sprayer element being disposed a sixth substantially predetermined distance away from a base of an inverted second container, the sixth substantially predetermined distance being substantially different from the fifth substantially predetermined distance;
said moving arrangement is further configured to move said sprayer element toward a base of an inverted second container through relative movement of said sprayer element and the inverted second container;
said moving arrangement is further configured to move said sprayer element to a seventh substantially predetermined distance away from a base of an inverted second container;
said discharging arrangement is further configured to discharge a second cleaning agent from said sprayer element;
said moving arrangement is further configured to move said sprayer element away from a base of an inverted second container;
said stopping arrangement is further configured to stop the discharge of a second cleaning agent from said sprayer element upon said sprayer element being disposed an eighth substantially predetermined distance away from a base of an inverted second container, the eighth substantially predetermined distance being substantially different from the seventh substantially predetermined distance;
a continuing arrangement being configured to continue the method with containers of the first physical configuration; and
said continuing arrangement is configured to continue the method with containers of the second physical configuration.

9. The method of cleaning containers in a container filling plant according to claim 6, wherein said method further comprises repeating steps B-K and steps M-V a plurality of times.

10. The method of cleaning containers in a container filling plant according to claim 9, wherein:
said first cleaning agent comprises water; and
said second cleaning agent comprises air.

11. The method of cleaning containers in a container filling plant according to claim 10, wherein:
said sprayer element is configured to move relative to said inverted first container; and
said sprayer element is configured to move relative to said inverted second container.

12. The method of cleaning containers in a container filling plant according to claim 11 including a substantially vertically adjustable actuator apparatus and a linear actuator, wherein:
said sprayer element is operatively connected to said substantially vertically adjustable actuator apparatus comprising a base and a control section;
said control section is configured to adjust the substantially vertical height of said base;
said control section is operatively connected to said linear actuator, which linear actuator is configured to adjust the substantially vertical height of said control section.

13. The method of cleaning containers in a container filling plant according to claim 12, wherein:
said sprayer element is configured to be moved, inside said inverted first container, to a portion of said inverted first container substantially immediately adjacent said base of said inverted first container; and
said sprayer element is configured to be moved, inside said inverted second container, to a portion of said inverted second container substantially immediately adjacent said base of said inverted second container.

14. The method of cleaning containers in a container filling plant according to claim 13, wherein said sprayer element comprises a nozzle configured to spray in all directions around said nozzle.

15. The method of cleaning containers in a container filling plant according to claim 14 including an inlet conveyor, an outlet conveyor, and a treatment conveyor, wherein:
said step of positioning a base of a first container above a mouth of said first container further comprises positioning said base of said first container above said mouth of said first container in said inlet conveyor;
said step of positioning a base of a second container above a mouth of said second container further comprises positioning said base of said second container above said mouth of said second container in said inlet conveyor; and
said method further comprises:
positioning said mouth of said first container above said base of said first container in said outlet conveyor;
positioning said mouth of said second container above said base of said second container in said outlet conveyor;
moving said inverted first container along said treatment conveyor between said inlet conveyor and said outlet conveyor; and
moving said inverted second container along said treatment conveyor between said inlet conveyor and said outlet conveyor.

16. A container cleaning arrangement configured to clean containers in a container filling plant; said container cleaning arrangement comprising:
a positioning arrangement being configured to position a base of a first container above the mouth of the first container, the first container comprising a first physical configuration;
an inserting arrangement being configured to insert a sprayer element into a mouth of an inverted first container through relative movement of said sprayer element and the inverted first container;
a moving arrangement being configured to move said sprayer element to a first substantially predetermined distance away from a base of an inverted first container;
a discharging arrangement being configured to discharge a first cleaning agent from said sprayer element;
said moving arrangement is further configured to move said sprayer element away from a base of an inverted first container;
a stopping arrangement being configured to stop the discharge of a first cleaning agent from said sprayer element upon said sprayer element being disposed a second substantially predetermined distance away from a base of an inverted first container, the second substantially predetermined distance being substantially different from the first substantially predetermined distance;
said moving arrangement is further configured to move said sprayer element toward a base of an inverted first container through relative movement of said sprayer element and the inverted first container;
said moving arrangement is further configured to move said sprayer element to a third substantially predetermined distance away from a base of an inverted first container;
said discharging arrangement being further configured to discharge a second cleaning agent from said sprayer element;
said moving arrangement is further configured to move said sprayer element away from a base of an inverted first container;
said stopping arrangement is further configured to stop the discharge of a second cleaning agent from said sprayer element upon said sprayer element being disposed a fourth substantially predetermined distance away from a base of an inverted first container, the fourth substantially predetermined distance being substantially different from the third substantially predetermined distance;
said positioning arrangement is further configured to position a base of a second container above the mouth of the second container, the second container comprising a second physical configuration, which second physical configuration is different than the first physical configuration;
said inserting arrangement is further configured to insert said sprayer element into a mouth of an inverted second container through relative movement of said sprayer element and the inverted second container;
said moving arrangement is further configured to move said sprayer element to a fifth substantially predetermined distance away from a base of an inverted second container;
said discharging arrangement is further configured to discharge a first cleaning agent from said sprayer element;
said moving arrangement is further configured to move said sprayer element away from a base of an inverted second container;
said stopping arrangement is further configured to stop the discharge of a first cleaning agent from said sprayer element upon said sprayer element being disposed a sixth substantially predetermined distance away from a base of an inverted second container, the sixth substantially predetermined distance being substantially different from the fifth substantially predetermined distance;
said moving arrangement is further configured to move said sprayer element toward a base of an inverted second container through relative movement of said sprayer element and the inverted second container;
said moving arrangement is further configured to move said sprayer element to a seventh substantially predetermined distance away from a base of an inverted second container;
said discharging arrangement is further configured to discharge a second cleaning agent from said sprayer element;
said moving arrangement is further configured to move said sprayer element away from a base of an inverted second container;
said stopping arrangement is further configured to stop the discharge of a second cleaning agent from said sprayer element upon said sprayer element being disposed an eighth substantially predetermined distance away from a base of an inverted second container, the eighth substantially predetermined distance being substantially different from the seventh substantially predetermined distance;
a continuing arrangement being configured to continue the method with containers of the first physical configuration; and
said continuing arrangement is configured to continue the method with containers of the second physical configuration.

17. The container cleaning arrangement according to claim 16, wherein said container cleaning arrangement is configured:
to move said sprayer element toward a base of an inverted first container a plurality of times; and
to move said sprayer element toward a base of an inverted second container a plurality of times.

18. The container cleaning arrangement according to claim 17, wherein:
said sprayer element is configured to discharge a first cleaning agent comprising water; and
said sprayer element is configured to discharge a second cleaning agent comprising air.

19. The container cleaning arrangement according to claim 18, wherein:
said sprayer element is configured to move relative to said inverted first container; and
said sprayer element is configured to move relative to said inverted second container.

20. The container cleaning arrangement according to claim 19 including a substantially vertically adjustable actuator apparatus, a linear actuator, an inlet conveyor, an outlet conveyor, and a treatment conveyor, wherein:
said sprayer element is operatively connected to said substantially vertically adjustable actuator apparatus comprising a base and a control section;
said control section is configured to adjust the substantially vertical height of said base;
said control section is operatively connected to said linear actuator, which linear actuator is configured to adjust the substantially vertical height of said control section;
said sprayer element is configured to be moved, inside said inverted first container, to a portion of said inverted first container substantially immediately adjacent said base of said inverted first container; and
said sprayer element is configured to be moved, inside said inverted second container, to a portion of said inverted second container substantially immediately adjacent said base of said inverted second container;
said sprayer element comprises a nozzle configured to spray in all directions around said nozzle;
said inlet conveyor is configured to position a base of a first container above a mouth of the first container;
said inlet conveyor is further configured to position a base of a second container above a mouth of the second container; and
said outlet conveyor is configured to position a mouth of a first container above a base of the first container;
said outlet conveyor is further configured to position a mouth of a second container above a base of the second container;
said treatment conveyor is configured to move an inverted first container between said inlet conveyor and said outlet conveyor; and
said treatment conveyor is further configured to move an inverted second container between said inlet conveyor and said outlet conveyor.
